(19) United States
(12) Patent Application Publication
(10) Pub. No.: US 2014/0048093 A1
(43) Pub. Date: Feb. 20, 2014

(54) Solid Coloring Compositions

(71) Applicants: Claire-Sophie Bernet, Bruxelles (Uccle) (BE); Anick Colson, Vedrin (BE); Serge Creutz, Liege (BE); Vishal Joshi, Gujarat (IN); Stephanie Postiaux, Ressaix (BE)

(72) Inventors: Claire-Sophie Bernet, Bruxelles (Uccle) (BE); Anick Colson, Vedrin (BE); Serge Creutz, Liege (BE); Vishal Joshi, Gujarat (IN); Stephanie Postiaux, Ressaix (BE)

(73) Assignee: DOW CORNING CORPORATION, Midland, MI (US)

(21) Appl. No.: 13/983,452

(22) PCT Filed: Dec. 21, 2011
(86) PCT No.: PCT/EP2011/073527

§ 371 Date: Nov. 5, 2013

(30) Foreign Application Priority Data
Mar. 28, 2011 (IN) 410/KOL/2011

(51) Int. Cl. A61K 8/891; A61K 8/898; A61Q 5/12; A61Q 5/08; A61Q 5/10

(52) U.S. Cl. CPC A61K 8/891; A61Q 5/08; A61Q 5/10; A61Q 5/12; A61K 8/898
USPC 132/208; 424/62; 8/405

(57) ABSTRACT

The present invention relates to solid colouring compositions for fibrous materials. The solid colouring compositions comprise a silicone in powder form and a solid colour modifying composition.

### CROSS-REFERENCE TO RELATED APPLICATIONS

This application claims the benefit of India patent application No. 410/KOL/2011 filed on Mar. 28 2011.

### BACKGROUND OF THE INVENTION

Colour modification of natural or synthetic fibres is useful to impart colour to textile fibre, or to modify hair colour.

Several different types of colour modifying agents may be used to modify the colour of fibres, among which natural dyes, synthetic dyes and bleaching agents. Among the synthetic dyes are oxidative dyes and non oxidative dyes.

Oxidative dyes, relying on precursors, are widely used as permanent fibre dyeing agents. Oxidation dye precursors in such fibre dyeing agents penetrate into fibre, and chemically impart a colour to the fibre by means of colour formation resulting from oxidative polymerisation under the action of an oxidation agent. Non-oxidation dyeing agents are used for semi-permanent or non-permanent fibre dyeing. Semi-permanent or non-oxidation dyeing agents are sometimes also referred to as direct dyes. Semi-permanent dyeing will usually colour fibre for up to six subsequent cleaning washes, such as shampoo, although a high proportion of colour is often lost after 2 or 3 washes. Semi-permanent fibre dyeing compositions are usually provided as single-component products, and may contain a variety of additives in addition to a direct dye.

It is desirable for the colour modifying composition containing an oxidation or non-oxidation dye precursor to be in the form of a powdery single-component product. Some of the oxidation dye precursors used for colour modification tend to quickly react together. When supplied as liquid form, they need to be supplied to the end-user in separate packaging, the end-user mixing them right before use in a specific ratio. Any divergence from the required ratio will lead to colour modification not corresponding to the targeted final colour. One way to prevent this is to provide the composition in solid form, which allows supplying all the reactive components in the right ratio as a one-part system while limiting the tendency for the chemicals to react together Accordingly, the end-user only needs to liquefy the composition in solid form by adding a solvent, such as water.

Colour modifying compositions, through the chemical reaction they involve, tend to partly damage the fibres, leading to unpleasant sensory feel and loss in shape and strength of the fibres. Regular subsequent washes, like shampooing of hair fibres, deteriorate the colour modification, leading to the need to repeat the colour modification process. Thus, there is a need to condition the fibres to improve the sensory feel upon and after application, the mechanical characteristics and the long-lastingness of the colour modification. This can be achieved by adding silicones, which may be liquids. However, if the silicones are added directly in the solid colour modifying composition, they will tend to spread upon storage. Accordingly, due to their usual hydrophobic nature, upon spreading, silicones will decrease the solubility of the solid colour modifying composition in typical solvent, such as water, which will lead to poor dissolution and formation of lumps and residues and thus uneven colour modification. Thus, there is a need to protect the silicone components during storage, while still releasing them during application. Finally, the conditioning solid composition needs to be versatile enough to be applicable across a range of compositions in solid form without having to change its composition.

There is still a need to provide fibre colour modifying compositions in solid form which do not form lumps on fibre and which are easy to rinse.

There is also still a need to provide conditioning to fibres such as hair when dyed or bleached using products in powder form. Conditioning of hair fibres includes ease to comb, shine, soft feel, colour maintenance.

### BRIEF SUMMARY OF THE INVENTION

The present invention relates to solid colouring compositions for fibrous materials. By colouring composition is generally meant a product which is designed to change the colour of fibrous materials, either by contribution of dyes or by bleaching. Fibres include but are not limited to keratinous fibres, for example hair, wool, and textile fibres, for example cotton.

The solid colouring compositions comprise a silicone in powder form. Using the silicone in powder form in the solid colouring composition provides protection upon storage, appropriate release during application, improved sensory feel upon and after application, improved mechanical characteristics and improved long-lastingness of the colour modification, convenience (easy to transport and allows ready to use one-part colour modifying composition with conditioning benefits), a new product format. Furthermore, a preservative is not mandatory.

Described herein is a solid colouring composition comprising

(A) a solid colour modifying composition
(B) a silicone in powder form.

The solid colour modifying composition (A) comprises at least one colour modifying agent such as a synthetic dye, a natural dye, a bleaching agent, or a mixture thereof, imparting a colour modification to the fibre to which it is applied.

The silicone in powder form (B) comprises at least one silicone polymer, a carrier and optionally a binder, wherein the polymer, carrier and optional binder are combined into a granular composition to result in the powder form. By powder form it is meant a matter in a finely divided state, a free flowing particulate matter, such as a granular composition or a granule which may result from an agglomeration or granulation process.

The composition in solid form is subsequently combined with a solvent, such as water, for application to the fibre in order to impart a colour change.

### DETAILED DESCRIPTION OF THE INVENTION

A wide variety of fibres may have its colour modified on purpose. Fibres may be of natural origin such as vegetal fibre and animal fibre, or of synthetic origin.

Fibres from vegetal origin include, but are not limited to, abaca, cotton, cellulose, linen.

Fibres from animal origin include, but are not limited to, silk, hair (human, camel, goat (mohair)), cashmere, wool (merino, llama, sheep, angora).

Fibres from synthetic origin include, but are not limited to, acetate, rayon, nylon, viscose, polyester, polyamide, microfiber, spandex (or elastane).

There are several different types colour modifying agents among which natural dyes, synthetic dyes, pigments and bleaching agents.

Natural dyes may be derived from plants, insects, animals or minerals. When extracted from plants, they may originate from the roots, the berries, the leaves, the wood.

Examples of natural dyes include those extracted from henna leaves, indigo leaves, camomile, curcuma roots, rhubarb, black alder tree bark (Rhamanus frangula L.), olive leaves, Canadian bloodroot, curcuma (Curcuma Longa L.), fustic, asafetida, mulberry, cutch, pomegranate, saffron, safflower, redwood, red sandalwood, haematoxylon wood (Haematoxylon campechianum L.), madder root (Rubia tinctorum L.), black elder, black apple berries, and mixtures thereof.

Synthetic dyes include fibre reactive dyes, direct dyes, vat dyes, naphthol dyes, sulphur dyes, all purpose dyes.

Fibre reactive dyes include dichlorotriazine, aminochlorotriazine (monochlorotriazine), aminofluorotriazine (monofluorotriazine), chlorodifluoropyrimidine, trichloropyrimidine, dichloroquinoxaline, sulfatoethylsulfone, sulfatoethylsulfonamide, bis(aminochlorotriazine), bis(aminonicotinotriazine.

Direct dyes include direct yellow 50, direct red 9, direct red 23, direct red 80, direct blue 98, direct blue 293, direct brown 116, direct black 22, direct black 80.

Vat dyes include vat blue 1 (indigo), vat blue 4, vat black 27, vat black 29, vat orange 16, vat yellow 33, vat green 1, vat green 3, vat brown 3, vat brown 72, vat violet 1, vat red 15, vat red 29.

Classification of hair fibre colouring systems is based on their lasting on hair, that is hair colour may be temporary, semi-permanent, demi-permanent or permanent. Temporary hair colour washes out in about two or three shampoos. Semi-permanent hair dye washes out in 7 to 12 shampoos. Demi-permanent hair colour washes out in about 28 shampoos, and permanent hair colour lasts until the hair has grown out or is cut.

Temporary hair colour modification is achieved by the deposition of a pigment on the hair fibre, there is no penetration of the pigment in the hair fibre. Temporary pigments include iron oxides, certified dye lakes, titanium dioxide coated micas, cationic dyes.

Semi-permanent hair colour relies on smaller dyes which penetrate the hair fibre. Semi permanent dyes include basic brown 17, basic brown 16, basic red 76, basic blue 26, basic blue 99, basic yellow 57, basic red 51, acid violet 43, HC yellow No. 2, HC yellow No. 5, HC red No. 3, HC blue No. 2, 4-hydroxypropylamino-3-nitrophenol, N,N-bis(2-hydroxyethyl)-2-nitro-p-phenylenediamine.

Permanent colouring of hair fibres may be achieved using oxidative dyes, which comprise primary intermediates (or precursors) and couplers, an oxidizing agent (usually hydrogen peroxide), and an alkalizing agent (usually ammonia).

Primary intermediates may be selected from para-phenylenediamine (PPD), 2-methyl-1,4-diaminobenzene, 2,6-dimethyl-1,4-diaminobenzene, 2,5-dimethyl-1,4-diaminobenzene, 2,3-dimethyl-1,4-diaminobenzene, 2-chloro-1,4-diaminobenzene, 2-methoxy-1,4-diaminobenzene, 1-phenylamino-4-aminobenzene, 1-dimethylamino-4-aminobenzene, 1-diethylamino-4-aminobenzene, 2-isopropyl-1,4-diaminobenzene, 1-hydroxypropylamino-4-aminobenzene, 2,6-dimethyl-3-methoxy-1,4-diaminobenzene, 1-amino-4-hydroxybenzene, 1-bis(beta-hydroxyethyl)amino-4-aminobenzene, 1-methoxyethylamino-4-aminobenzene, 2-hydroxymethyl-1,4-diaminobenzene, 2-hydroxyethyl-1,4-diaminobenzene, p-phenylenediamine, p-aminophenol, o-aminophenol, N,N-bis(2-hydroxyethyl)-p-phenylenediamine, 2,5-diaminotoluene, 5.6 dihydroxyindole, and derivatives thereof, their salts and mixtures thereof and various types of pyrimidines such as 2,3,4,5-tetraminopyrimidine sulfate and 2,5,6-triamino-4-pyrimidinol-sulfate.

Couplers may be selected from meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methylpyrazolone, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene. 6-ethoxy-1-bis(beta-hydroxyethyl)amino-3-aminobenzene, 6-hydroxyethyl-1,3-diaminobenzene, 3,4-methylenedioxyphenol, 3,4-methylenedioxy-1-[(beta-hydroxyethyl) amino]benzene, 1-methoxy-2-amino-4-[(beta-hydroxyethyl)amino]benzene, 1-hydroxy-3-(dimeth-ylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-am inobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamioniphenoxyethanol, and mixtures thereof.

Demi permanent hair colour is permanent hair colour that contains an alkaline agent other than ammonia (e.g., ethanolamine, sodium carbonate) and, while always employed with a developer, the concentration of hydrogen peroxide in that developer may be lower than that used with a permanent hair colour.

Another type of colour modifying agent is a bleaching agent, or bleach, which is used to removes colours or whiten the fibre. Bleaching agents may be oxidizing bleach, which is breaking the chemical bonds that make up the chromophore into a different substance that either does not contain a chromophore, or contains a chromophore that does not absorb visible light. They may be reducing bleach, converting double bonds in the chromophore into single bonds, so eliminating the ability of the chromophore to absorb visible light.

Common bleaching agents include sodium hypochlorite (NaClO), hydrogen peroxide or a peroxide-releasing compound such as sodium perborate, sodium percarbonate, sodium dithionite, sodium persulfate, tetrasodium pyrophosphate, or urea peroxide. Other bleaching agents include chlorine dioxide, benzoyl peroxide, sodium thiosulfate or peracetic acid. Bleaching powder includes calcium hypochlorite.

Mixtures of bleaching agents and dyes may be used.

The colour modifying agent is provided in solid form, typically as a powder. Particle sizes of colour modifying agents are in the range of 0,0001 mm to 3 mm. Particle sizes will be impacted by the origin of the powder and its treatment such as milling. Commercial bleaching agents typically have a particle size in the range of 0,01 mm to 3 mm. Dyes typically have a particle size in the range of 0,0001 mm to 1 mm.

The silicone composition in powder form comprises a carrier, a silicone polymer and optionally a binder. The silicone polymer is liquefied to produce a liquid silicone containing composition and then the liquid silicone containing composition is applied to the carrier and subsequently solidified either by drying or cooling, producing the powder form. The liquid silicone containing composition comprises at least one silicone polymer in the form of a neat polymer or in the form of an emulsion.

The carriers may be solid particulate carriers, of natural or synthetic origin, and include:
silicates and aluminasilicates such as zeolites, magnesium silicate, calcium silicate, sodium silicate, mica, bentonite, diatomite, sepiolite, natural or modified clays, talc;
neat or treated silica;
carbohydrates such as lactose, dextrose, maltodextrin, alginate, chitin, chitosan, starch, wood flour, cellulose, cellulose derivatives such as sodium carboxymethylcel lulose;
calcium sulphate, sodium sulphate, magnesium sulphate, calcium carbonate, sodium acetate, sodium bicarbonate, sodium perborate, sodium citrate, phosphates such as sodium tripolyphosphate.

The carrier may comprise a mixture of different carriers. The carrier may be free of silica.

The mean particle size of a solid particulate carrier is typically in the range of 0.001 mm to 0.250 mm, alternatively 0.001 mm to 0.100 mm, alternatively 0.002 mm to 0.015 or 0.030 mm.

The optional binder may be used to improve the storage stability of the granules or trigger the release of the silicone. The binder may be water-soluble or water-dispersible; anionic, cationic or non ionic. The binder may be a linear polymer, a branched polymer, or a partially cross-linked polymer.

Examples of binders include:
polycarboxylate binders which are water soluble polymers such as polymerised products of unsaturated monomeric acids, e.g. acrylic acid, maleic acid, maleic anhydride, fumaric acid, itaconic acid, aconitic acid, mesaconic acid, citraconic acid and methylenemalonic acid;
polyoxyalkylene polymers such as polyethylene glycol;
carbohydrates such as polysaccharides, cationic starch, sugar syrup binders, malitol syrup, maltodextrin solution, water-soluble or water-swellable cellulose derivatives such as sodium carboxymethylcellulose, hydroxyethyl cellulose, hydoxypropylcellulose quaternized with glycidyl C₁₂-C₂₂ alkyl dimethyl ammonium chloride, cationic hydroxyalkyl cellulose including those with the CTFA designation Polyquaternium10, Polyquaternium 67, Polyquaternium 4;
homopolymers or copolymers prepared from monoethylenically unsaturated monomers, i.e., allylic and vinyl monomers. In particular, the binder is a homopolymer or copolymer prepared from acrylic or methacrylic monomers. Some examples of monomers that may be used to prepare the homopolymer or copolymer include dialkylaminoalkyl acrylates, dialkylaminoalkyl methacrylates, dialkylaminoalkyl acrylamides, dialkylaminoalkylalkyl acrylamides, dialkylaminoalkyl methacrylamides, dialkylaminoalkylalkyl methacrylamides, in which the alkyl groups are alkyl groups containing 1-4 carbon atoms, vinylpyridine, vinylimidazole; wherein the monomers may be partially quaternized, fully quaternized, or salified, by an acid, a quaternizing agent, benzyl chloride, methyl chloride, an alkyl chloride, an aryl chlorides, or dimethylsulfate. As used herein, salified refers to the salt formed by the acid-base reaction between the amino and an acid.

The binder may be mixed with the liquid silicone containing before being deposited on the carrier, or alternatively is separately deposited on the carrier either at the same time or subsequently, or at both times. In both cases, to ensure even deposition on the carrier, the binder should be liquid. Typically to liquefy the binder it is solubilised in a solvent or it is melted. The binder component may be used at 0.1 to 20% by weight of the silicone in powder form (B), alternatively 0.2 to 15% and alternatively 0.5 to 10%.

The silicone polymer in the liquid silicone containing composition may be in the form of a neat polymer or in the form of an emulsion. Any combination or mixture of different silicones may also be used.

Silicone polymers are known in the art as are methods for making them and many of them are commercially available.

Silicone polymers may be classified by their viscosity, their volatility, their molecular weight, the presence of organofunctional groups, the presence of crosslinking, and by many other means.

Silicone polymers modified with organofunctional groups are silicones containing in their structure one or more organofunctional groups attached via a Si-C or Si-O-C linkage.

Suitable organofunctional groups include alkyl groups (such as methyl, ethyl, propyl, butyl, nonyl, dodecyl, tetradecyl, hexadecyl groups), unsaturated alkenyls or alkynyls (such as vinyl, allyl, hexenyl groups), aryl groups (such as phenyl), amine groups, amide groups, imine groups, imide groups, polyether groups, amido polyether groups, quaternary ammonium groups, saccharide groups, amino acids, hydroxyl groups, hydrocarbyl groups, carboxyl groups, carboxy polyether groups, hydrogen, fluoro groups, acrylic groups, epoxy groups, mercapto groups, etc.

The organofunctional groups may be located in pendant or terminal positions of the silicone polymer, or in both terminal and pendant positions.

The silicone polymers classified as volatile include those having a boiling point below 250° C., such as (i) cyclic silicones containing from 3 to 7 and typically from 5 to 6 silicon atoms; (ii) linear volatile silicones having 2 to 9 silicon atoms and having a viscosity of less than or equal to 5 mm²/s at 25° C. The volatile silicones may also be mixtures of (i) and (ii).

The silicone polymers classified as non-volatile silicones include polyalkylsiloxanes, polyalkylarylsiloxanes, silicone gums, silicone resins, silicone elastomers, a variety of silicone modified with organofunctional groups.

Polyalkylsiloxanes include polydimethylsiloxanes and polydiethylsiloxanes.

Polydimethylsiloxanes include those silicones that typically contain trimethylsilyl end groups (CTFA designation dimethicone) having a viscosity of from 5 mm²/s to 2.5 million mm²/s at 25° C., and typically 10 to 1 million mm²/s. Also suitable polyalkylsiloxanes are polydimethylsiloxanes hydroxylated at the terminal end of the chain (CTFA designation dimethiconol).

Polyalkylarylsiloxanes include linear and branched polydimethylmethylphenylsiloxanes and polydimethyldiphenylsiloxanes with a viscosity of from 10 to 50 000 mm²/s at 25° C.

Silicone gums include polydiorganosiloxanes having high number-average molecular masses of between 200,000 and 1,000,000. To make the silicone gums easier to handle and able to mix with the solid particulate carrier they are typically used in conjunction with a solvent. This solvent may be chosen from volatile silicones, polydimethylsiloxane oils, isoparaffins, hydrocarbon solvents, or mixtures thereof. Mixtures of silicone gum and solvent include those formed from a polydimethylsiloxane hydroxylated at the terminal end of the chain (CTFA designation dimethiconol) and from a cyclic polydimethylsiloxane (CTFA designation cyclomethicone).

Silicone resins are crosslinked siloxane systems and consist of siloxane units of the general formula RₑSiO_{4-e/2} wherein R denotes a hydrocarbon-based group having from 1 to 16 carbon atoms or a phenyl group and wherein e may have a value of from 0 to 3, but typically has an average value of from 0.5 to 2. The degree of crosslinking required to obtain a suitable silicone resin will vary according to the specifics of the silane monomer units incorporated during manufacture of the silicone resin. Among these products, those particularly typical are the ones in which R denotes a C₁C₄ lower alkyl radical, more particularly methyl, or a phenyl radical. The organopolysiloxane resins may be used alone or in conjunction with a solvent. Such solvent may be chosen from volatile silicones, polydimethylsiloxane oils, isoparaffins, hydrocarbon solvents, or mixtures thereof. Mixtures may be formed from a silicone resin (CTFA designation trimethylsiloxysilicate) and a cyclic or linear polydimethylsiloxane (CTFA designation cyclomethicone or dimethicone) or phenyltrimethylsiloxysilane.

Silicone resins include those described in U.S. Pat. No. 5,152,984 and U.S. Pat. No. 5,126,126, such as aminopropyl phenyl trimethicone (CTFA designation).

Silicone elastomers are another type of crosslinked siloxane systems. Most of these elastomers can be used to cause volatile silicones fluids or low polarity organic solvents such as isododecane to gel. They may be in the form of solid particles, spherical or non spherical, or in the form of swollen gels, where the silicone elastomer is combined with a solvent such as volatile silicones, polydimethylsiloxane oils, isoparaffins, hydrocarbon solvents, or mixtures thereof. Representative examples of such silicone elastomers are taught in U.S. Pat. No. 5,880,210 and U.S. Pat. No. 5,760,116. To improve the compatibility of silicone elastomers with various personal care ingredients, alkyls, polyether, amines or other organofunctional groups may be grafted onto the silicone elastomer backbone. Representative examples of such organofunctional silicone elastomers are taught in U.S. Pat. No. 5,811,487, U.S. Pat. No. 5,880,210, U.S. Pat. No. 6,200,581, US5,236,986, U.S. Pat. No. 6,331,604, U.S. Pat. No. 6,262,170, U.S. Pat. No. 6,531,540, U.S. Pat. No. 6,365,670, WO2004/104013 and WO2004/103326.

Silicone polymers modified with organofunctional groups include those having polyether groups and amine groups.

Silicone polyethers are silicone polymers containing polyether groups, also referred to as poly(oxyalkylene) group, such as polyethylenoxy and/or polypropylenoxy groups optionally containing C₆-C₂₄ alkyl groups. They may be water soluble or water dispersible. They may be linear rake or graft type materials, or ABA and ABn types where the B is the silicone polymer block, and the A is the poly(oxyalkylene) group. The poly(oxyalkylene) group may consist of polyethylene oxide, polypropylene oxide, or mixed polyethylene oxide/polypropylene oxide groups. Other oxides, such as butylene oxide or phenylene oxide are also possible. They include the products known as PEG/PPG-dimethicone and (C₁₂)alkylmethicone copolyol.

Amino functional silicones are silicone polymers containing substituted or unsubstituted amine groups, such as aminopropyl, aminoethylaminopropyl, aminoethylaminoisobutyl groups.

Silicone polymers modified with organofunctional groups additionally include those having
alkoxylated groups;
hydroxyl groups such as the polyorganosiloxanes containing a hydroxyalkyl function, as described in EP1081272, U.S. Pat. No. 6,171,515 and U.S. Pat. No. 6,136,215;
bis-hydroxy/methoxy amodimethicone;
amino-acid functional silicones, such as those obtained by reacting an amino acid derivative selected from the group of an N-acyl amino acid and an N-aroyl amino acid with an amino functional siloxane, further described in WO2007/141565;
quaternary ammonium functional silicones, such as those described in U.S. Pat. No. 6,482,969 and U.S. Pat. No. 6,607,717, such as silicone quaternium-16 (CTFA designation) or monoquaternary ammonium functional derivatives of alkanolamino polydimethylsiloxanes, such as disclosed in U.S. Pat. No. 5,026,489;
hydrocarbyl functional silicones such as those comprising a siloxy unit of the formula R¹R′ᵢSiO_{(3-i)/2} wherein R′ is any monovalent hydrocarbon group, but typically is an alkyl, cycloalkyl, alkenyl, alkaryl, aralkyl, or aryl group containing 1-20 carbon atoms, R¹ is a hydrocarbyl group having the formula —R²OCH₂CH₂OH, wherein R² is a divalent hydrocarbon group containing 2 to 6 carbon atoms and i has a value of from zero to 2, further described in U.S. Pat. No. 2,823,218, U.S. Pat. No. 5,486,566, U.S. Pat. No. 6,060,044 and US20020524, such bis-hydroxyethoxypropyl dimethicone (CTFA designation).

Silicone polymers modified with organofunctional groups further include copolymers formed by a silicone block and an organofunctional block in an ABA or ABn type of structure, such as:
amino ABn silicone polyether block copolymer, such as those where an amino functionality is added to the ABn silicone polyether copolymer, also described in IP.COM 00141525 such as bis-isobutyl PEG/PPG-20/35/amodimethicone copolymer (CTFA designation);
siloxane-based polyamide such as disclosed in U.S. Pat. No. 6,051,216, and their variants such as silicone polyether-amide block copolymers such as disclosed in US2008/0045687
vinyl-type polymers such as disclosed in EP0963751, having a carbosiloxane dendrimer structure on their side molecular chain, which may be used as neat polymer or as a solution or a dispersion in a liquid such as a silicone oil, organic oil, alcohol, or water, such as acrylates/polytrimethylsiloxymethacrylate copolymer (CTFA designation)
saccharide siloxane copolymers (copolymer) such as those having the following formula:

R⁴ₐR³₍₃₋ₐ₎SiO—[(SiR⁴R³O)ₘ—(SiR³₂O)ₙ]_{y}—SiR³₍₃₋ₐ₎R⁴ₐ; where:
each R³ can be the same or different and each R³ comprises hydrogen, an alkyl group of 1 to 12 carbon atoms, an organic group, or a group of formula R⁵-Q;
Q comprises an epoxy, cycloalkylepoxy, primary or secondary amino, ethylenediamine, carboxy, halogen, vinyl, allyl, anhydride, or mercapto functionality;
subscripts m and n are integers from 0 to 10,000 and may be the same or different;
each subscript a is independently 0, 1, 2, or 3;
subscript y is an integer such that the copolymer has a molecular weight less than 1 million;
each R⁴ has formula Z-(G¹)_{b}-(G²)_{C}, and there is an average of at least one R⁴ per copolymer molecule, where:
   G¹ is a saccharide component comprising 5 to 12 carbon atoms,
   a quantity (b+c) has a value ranging from 1 to 10, and subscript b or subscript c can be 0,
   G² is a saccharide component comprising 5 to 12 carbon atoms additionally substituted with organic or organosilicon radicals,
   each Z is a linking group and is independently selected from the group consisting of:
      —R⁵—N(R¹⁰)—C(O)-R⁶—, —R⁵—CH(OH)—CH²—N(R¹⁰)—R⁶—,
      or
      —R5—CH(N(R6)(R10))CH2OH; where each R⁵ and each R⁶ are divalent spacer groups comprising a group of formula (R⁷)r(R⁸)s(R⁹)t, where at least one of subscripts r, s and t is 1, and each R⁷ and each R⁹ are independently either an alkylene group of 1 to 12 carbon atoms or a group of formula (R¹¹O)ₚ, where: subscript p is an integer with a value ranging from 1 to 50, and each R¹¹ is a divalent organic group, and each R¹¹O may be the same or different, each R⁸ is —N(R¹⁰)—, where: R¹⁰ is selected from R⁵, a group of formula Z-X, an unsaturated hydrocarbon group, or a reaction product of —N(H)— with an epoxy functional group, a cycloalkylepoxy functional group, a glycidyl ether functional group, an acidic anhydride functional group, or a lactone; each X is independently a divalent a carboxylic acid, phosphate, sulfate, sulfonate or quaternary ammonium radical, and with the provisos that at least one of R⁵ and R⁶ must be present in the linking group, and each R⁵ and each R⁶ may be the same or different ionically-modified saccharide-siloxane copolymer, such as described in WO2006/127924.

Silicone polymers modified with organofunctional groups further include alkylmethylsiloxane materials which exist under the form of liquids or waxes. In liquid form they can be either cyclic having a structure comprising:

[MeR12SiO]ₛ[Me₂SiO]_{q}

or linear having a structure comprising

R¹³Me₂SiO(MeR¹²SiO)_{W}(Me₂SiO)_{X}SiR¹³Me₂

wherein each R¹² is independently a hydrocarbon of 6 to 30 carbon atoms, R¹³ is methyl or R¹², s is 1-6, q is 0-5, w is 0-5 and x is 0-5, provided s+q is 3-6 and q is not 0 if R¹³ is methyl. These liquids may be either volatile or non-volatile and they can have a wide range of viscosities such as from about 0.65 to about 50,000 mm²/s at 25° C. Alkylmethylsiloxane waxes have the structure:

R¹³Me₂SiO(Me₂SiO)_{g}(MeR¹²SiO)_{Z}SiMe₂R¹³

wherein g is 0-100, z is 1-100, R¹² is an alkyl group of 6-30 carbon atoms and R¹³ is methyl or R¹². Typically, the alkylmethylsiloxane has the formula:

Me₃SiO(Me₂SiO)_{g}(Me R¹²SiO)_{Z}SiMe₃

Emulsions of neat polymers described above may also be used. Emulsions of silicone polymers such as dimethicone, dimethiconol, amino functional silicone, divinyldimethicone/dimethicone copolymer (CTFA designation) and other silicone polymers exist and are described in the art.

The emulsions are based on surfactants which may be non ionic, cationic, anionic or combinations thereof. Their particle sizes may be in the range of 1 nm to 1 mm, alternatively 10 nm to 0.1 mm. They may be transparent, translucent or opaque.

Silicone polymers which may be used in (B) may be non elastomeric, and are further exemplified by polyalkylsiloxanes containing trimethylsilyl end groups; polyalkylsiloxanes containing dimethylsilanol end groups; silicone polymers modified with organofunctional groups such as aryl groups (such as phenyl), amine groups, polyether groups, quaternary ammonium groups, saccharide groups, amino acids, vinyl groups, hydroxyl groups; and mixtures or emulsions of these. Silicone polymers which may be used in (B) typically exclude silicone elastomers.

The solid colouring composition may also contain optional ingredients. The optional ingredients may be added to A, to B or to the combination of (A) and (B).

Optional ingredients include perfumes, essential oils, deposition agents, buffering agents, pH adjusting agents, stabilizers, pigments, amino-acid derivatives, proteins, ceramides, preservatives, anti-dandruff agent, disinfectants, glycols, vitamins and/or their derivatives, provitamins, styling agents, sunscreen agents, humectants, water-soluble emollients, oil components, emollients, esters, soothing ingredients, antiperspirants, malodor sequestrants, surfactants, antioxidants, natural herbs, antimicrobial agents, hair growth enhancers.

Deposition agents may be used to improve the deposition of the silicone onto the fibres and potentially providing more fibre conditioning such as hair grooming. They are typically in the form of organic cationic agents.

Deposition agents include cationic surfactants such as cetyl trimethylammonium chloride, cetyl trimethylammonium bromide, and stearyltrimethylammonium chloride; polysaccharide polymers, such as cationic cellulose derivatives, cationic starch derivatives, cationic guar gum derivatives such as guar hydroxypropyltrimonium chloride.

Specific deposition agents include those having CTFA designations Polyquaternium-6, Polyquaternium-7, Polyquaternium-16, Polyquaternium-8, Polyquaternium-10, Polyquaternium-11, and Polyquaternium-23.

Proteins include those extracted from wheat, soy, rice, corn, keratin, elastin or silk.

Most are in the hydrolyzed form and they may also be quaternised to provide better performance.

Perfumes are fragrant odoriferous substance or mixtures of fragrant odoriferous substances including natural substances obtained by extraction of flowers, herbs, leaves, roots, barks, wood, blossoms or plants; artificial substances including mixtures of different natural oils or oil constituents; and synthetically produced substances. Some examples of perfume ingredients include hexyl cinnamic aldehyde; orange oil; lemon oil; grapefruit oil; bergamot oil; clove oil; dodecalactone gamma; geraniol; linalool; amyl cinnamic aldehyde; amyl salicylate; hexyl salicylate; terpineol; para-methoxyacetophenone; para-methoxy-alpha-phenylpropene; methyl- 2-n-hexyl-3-oxo-cyclopentane carboxylate; and undecalactone gamma.

pH adjusting agents may be used to adjust pH of the liquid silicone containing composition, within the range of 4 to 9 alternatively within the range of 5 to 7. Any water soluble acid such as a carboxylic acid or a mineral acid is suitable. Suitable acids include mineral acids such as hydrochloric acid, sulphuric acid, and phosphoric acid, monocarboxylic acid such as acetic acid and lactic acid, and polycarboxylic acids such as succinic acid, adipic acid, and citric acid.

Pigments include iron oxides and titanium dioxide.

Preservatives include parabens and derivatives, BHT, BHA, DMDMH.

Vitamins include lipid-soluble vitamins and their derivatives and water-soluble vitamins and their derivatives. Lipid-soluble vitamins include retinol (vitamin A), ergocalciferol (vitamin D₂), cholecalciferol (vitamin D₃), phytonadione (vitamin K₁), and tocopherol (vitamin E). Water-soluble vitamins include ascorbic acid (vitamin C), thiamin (vitamin B₁) niacin (nicotinic acid), niacinamide (vitamin B₃), riboflavin (vitamin B₂), pantothenic acid (vitamin B₅), biotin, folic acid, pyridoxine (vitamin B₆), and cyanocobalamin (vitamin B₁₂).

Provitamins may also be used, such as panthenol.

Hair growth enhancers include Gotu kola, Gingko biloba, Aloe vera, niacin, Cayenne Pepper (Capsicum), ginseng extract, copper peptides, retinoic acid, minoxidil and minoxidil-like agents, DHT blockers such as finasteride and anti-androgens, and hair growth enhancers based on copper-peptides.

Water-soluble emollients include lower molecular weight aliphatic diols such as propylene glycol and butylene glycol; polyols such as glycerine and sorbitol; and polyoxyethylene polymers such as polyethylene glycol 200.

Oil components include organic butters such as mango, cocoa, shea butters.

Natural herbs or extracts include aloe vera, amla (Emblica officinalis), bhringaraj (Elcipta alba), brahmi (Bacopa monniera or Centella asiatica), Cassia obovata, curry, fennel, ginseng, lotus, Multani mitti or Fuller's Earth, neem (Azadirachta indica), orange peel powder (Citrus sinensis), reetha powder (soap nut), rose petal powder, shikakai (Acacia cancinna), tulsi (Ocimum sanctum).

The process for producing (B) may be referred to as granulation or agglomeration. The liquid silicone containing composition is contacted with the carrier in a mixer in which droplets of the liquid silicone containing composition become agglomerated with the carrier, resulting in the silicone in powder form (B). The silicone in powder form may also be referred to as a granular composition or granules.

In producing (B) contact may for example be in a granulating mixer, an extruder, a compactor or in a high shear or low shear mixer. Typically the liquid silicone containing composition is contacted with the carrier in a granulating mixer in which the agglomerated product is kept in powder form. The granulating mixer is generally a high shear mixer such as an Eirich (trade mark) pan granulator, a Schugi (trade mark) mixer, a Pin mixer, a Glatt (trade mark) mixer, a Paxeson-Kelly (trade mark) twin core blender, a Lodige ploughshare mixer, an Aeromatic (trade mark) fluidized bed granulator or a Pharma (trade mark) drum mixer. In most granulating mixers, the liquid silicone containing composition is sprayed onto the carrier particles while the carrier is being agitated. The liquid silicone containing composition may alternatively be poured into the mixer instead of spraying.

The resulting granular composition is collected from the granulating mixer and packaged. The product from a vertical continuous granulating mixer may be fed to a fluidised bed which cools and/or dries the granules and fluidises them for transport to a packing station. If the particle size distribution of granules at the outlet of the granulating mixer is larger than desired, including fines and oversize material, the fines can for example be recovered in a filter coupled with the fluidized bed cyclone and/or in a classification unit and recycled with fresh particles feeding the mixer, and oversize material can be collected, crushed down and mixed with the granular composition in a fluidized bed.

If the liquid silicone containing composition and the carrier are agglomerated in an apparatus which does not maintain the agglomerated mixture as separate granules, for example an extruder or a compactor, the agglomerated mixture can be converted into granules by flaking, by comminuting an extruded strand or by spheronization after extrusion.

One typical form of granulating mixer is a vertical continuous granulating mixer comprising blades rotating within a tubular housing and having an inlet for the carrier and a spray inlet for the liquid silicone containing composition to contact the carrier above the blades. The blades are mounted on a substantially vertical shaft aligned with the housing and rotating within the housing. The blades have a predetermined clearance from the inner wall of the housing. Contact with the liquid silicone containing composition agglomerates the carrier into granules; the liquid silicone containing composition acts as a binder by absorbing the kinetic energy of colliding particles of the carrier. The blades maintain the solid particles and granules in motion and prevent agglomeration into granules which are too large. Examples of such vertical continuous granulating mixers are described in U.S. Pat. No. 4,767,217, EP744215 and WO03/059520. Vertical continuous granulating mixer technology has the advantage that the residence time in the mixing chamber is very short, for example about 1 second, giving the possibility of high throughput.

The ratio of the weight of liquid silicone containing composition to the weight of carrier in the silicone in powder form (B) can be varied within wide limits. Generally this ratio is at least 1:99 and may be up to 50:50 or even higher provided that the granules produced are stable and do not agglomerate further under the forces to which they are subjected while being transported. Typically the ratio of the weight of liquid silicone containing composition fed to the mixer to the weight of carrier fed to the mixer is in the range 15:85 to 50:50.

Accordingly, the weight ratio of silicone polymer to carrier in the silicone in powder form produced after drying is in the range 2:98 to 40:60, alternatively in the range 4:96 to 25:75 or, alternatively in the range 25:75 to 35:65.

The silicone in powder form (B) has a mean particle size typically in the range of 0.02 mm to 1.50 mm, alternatively 0.05 mm to 1.00 mm, alternatively 0.05 mm to 0.70 mm.

The method of making the solid colouring composition comprises the steps of:
Producing a silicone in powder form by contacting the liquid silicone containing composition with the carrier in a mixer in which droplets of the liquid silicone
Combining the silicone in powder form (B) with the solid colour modifying composition (A).

The ratio of silicone in powder form (B) to the solid colour modifying composition (A) in the solid colouring composition is in the range of 1:99 to 90:10, alternatively 5:95 to 30:70.

The solid colouring composition is used to modify the colour of fibre. The method of modifying the colour of fibre comprises the steps of:
Providing a solid colouring composition comprising (A) and (B)
Mixing the solid colouring composition with water to form a water mixture
Applying the water mixture to a fibre
Rinsing the fibre.

The solid colouring composition is used upon mixing with water, at a ratio of solid colouring composition to water of 1:5 to 1:12, alternatively 1:7 to 1:10, alternatively 1:9. The water mixture may be applied to the fibre immediately after mixing or may be left to stand for a time of 1 minute to 4 hours before being applied to the fibre.

The amount of water mixture applied to the fibre will vary depending on the type of fibre and the result to be achieved. One skilled in the art would be able to determine the amount based on the level of colour modification desired. For example, when the fibre is hair, the ratio of water mixture to hair fibre is in the range of 10:1 to 1:100.

After the water mixture has been applied to the fibre, it may be left to stand on the fibre for a time of 5 minutes to 4 hours or more. One skilled in the art would be able to determine the time based on the level of colour modification desired. The fibre is then rinsed, typically with water. Shampoo or detergents may be used during the rinse to facilitate the removal of an excess of colour modifying agent. The amount of time to rinse fibre will depend on the type and amount of fibre, and the colour modifying agent in (A). Typically rinse times are 1 minute to several hours. Some types of fibres may need several rinse cycles. One skilled in the art will know how long and how many rinse cycles are needed depending on the fibre and colour modifying agent considered.

The silicone in the composition in solid form provides several benefits to the colour modifying composition such as:
Fibre conditioning such as improved wet and dry feel, smoothness, softness, slipperiness, reduced drying time, colour protection/retention
When the fibre is hair, benefits include wet and dry detangling and combing, reduced flyaway/decreased static, body, volume, moisturisation, fullness, frizz control, shine/lustre, heat protection, strengthening, styling.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus may be considered to constitute typical modes for its practice. However, those of skill in the art should, in light of the present disclosure, appreciate that many changes may be made in the specific embodiments which are disclosed and still obtain a like or similar result without departing from the spirit and scope of the invention. All percentages are in wt. %.

Caucasian slightly bleached hair under the form of 2 g round tresses of 25 cm length, provided by International Hair Importers, US, were used. Indian virgin dark hair tresses also under the form of 2 g round tresses of 25 cm length, provided by a local Indian supplier were used.

Natural henna based powder from Godrej (commercial): “Nupur 100% Natural Mehendi” contains a mixture of Lawsonia inermis leaf powder (Mehendi), Aloe barbadensis leaf powder (Aloe Vera), Azadirachta indica leaf powder (Neem), Centella asiatica leaf extract (Brahmi), Eclipta alba powder (Bhringraj), Emblica officinalist fruit extract (Alma), Hibiscus rosasinensis flower powder (Jaswand), Acacia concinna pod powder (Shikakai), Nardostachys jatamansi rhizome powder (Jatamansi) and Trigonella foenum-graecum seed powder (Methi). The particle size of the powder is below 0.2 mm.

Bleaching agent powder Eau Ecarlate from Spotless Benelux (commercial): “SOS Linge deteint” (decoloured wash) contains a mixture of more than 30% wt sodium dithionate, sodium carbonate, parfums (linalool). The particle size of the powder is below 0.5 mm.

Testing on hair was carried out for the parameters of detangling ease, shine, sensory feel, colour and colour retention. Significant differences are set at a confidence level of at least 95%.

Wet combing test: panellists are asked to disentangle tresses while time is measured. The average recorded times are given under the corresponding compositions.

Sensory test—triangular: 3 hair tresses (2 the same and 1 different) are submitted to panellists who are requested to find the different tress compared to the 2 others. Comparison was run vs the Control tress without silicone treatment.

Shine test—pair comparison: 2 tresses are shown panellists who are requested to indicate which is shinier than the other. Comparison was run vs the Control tress without silicone treatment.

Colour test: the colour of the coloured tresses was measured using a colourimeter, ColourSphere BYK Gardner. Comparison was run vs the Control tress without silicone treatment.

Colour loss test (colour retention test): the colour of the coloured tresses was measured using a colourimeter, ColourSphere BYK Gardner, before and after washes using a sodium laureth sulphate dispersion at 9% wt active. A colour difference of 1 unit is considered to be a perceivable difference for the human eye.
Measure colour after colouring—before washes
Wash tresses 5 times with sodium laureth sulphate dispersion at 9% active
Measure colour after the 5 washes
Calculate Delta E between before and after washes—colour loss:

ΔE*=√(L₁−L₂)²+(a₁−a₂)²+(b₁−b₂)²

### Preparation Example 1

A liquid solution containing a silicone polymer is poured very slowly into a high shear mixer in which the carrier is placed. The mixture is stirred continuously until a granular composition is obtained. The granular composition is then passed over an Aeromatic spray granulator for 10 minutes at 60° C. The silicone compositions in powder form are described in Table 1. The compositions are indicated in percentage by weight.

The granular compositions were stable in storage for at least 4 weeks.

TABLE 1

| Silicone | | | Silicone |
|---|---|---|---|
| compositions in | | | content in |
| powder form (SCPF) | Carrier | Silicone type | SCPF |
| SCPF 1 | Corn Starch | Dimethicone (60 000 cSt) emulsion | 19% |
| SCPF 2 | Corn Starch | Cationic emulsion of amino functional | 13.4% |
| | | silicone fluid | |
| SCPF 3 | Corn Starch | Dimethicone (500 000 cSt) emulsion | 24.2% |
| SCPF 4 | Corn Starch | Microemulsion of amino functional | 12.2% |
| | | silicone fluid | |
| SCPF 5 | Corn Starch | Dimethicone (60 000 cSt) emulsion | 20% |
| SCPF 6 | Corn Starch | Dimethicone (500 000 cSt) emulsion | 20% |
| SCPF 7 | Corn Starch | Phenyltrimethicone fluid | 20% |
| SCPF 8 | Corn Starch | Aminopropyl phenyl trimethicone fluid | 20% |
| SCPF 9 | Corn Starch | Dimethicone (500 000 cSt) emulsion | 10% |
| SCPF 10 | Corn Starch | Cationic emulsion of amino functional | 10% |
| | | silicone fluid | |
| SCPF 11 | Corn Starch | Silicone quat microemulsion | 10% |
| SCPF 12 | Corn Starch | Cationic emulsion of slightly | 11.6% |
| | | crosslinked silicone polymer | |

### Examples 1 TO 4 and Comparative Example 1

The natural henna based powder described above was mixed with SCPF produced in Preparation Example 1 in the amounts as disclosed in Table 2, using the following procedure:
Blend the natural henna powder hair colourant and silicone compositions in powder form together at amount disclosed in Table 2
Add water @37° C. and mix with the spatula
Soak this preparation in water for 2 hours and a half
Apply 5 g of mixture per hair tress
Leave on hair during 2 hours and a half
Rinse each tress with water @37° C. during 1 min 30 s
For detangling test: proceed to the evaluation and let dry for 1 night for other testings
For other uses: let dry for 1 night

TABLE 2

| | | | | Silicone |
|---|---|---|---|---|
| | Henna | SCPF | Water | active |
| | content | content | content | content |
| Comparative | 12% | 0% | 88% | 0% |
| Example 1 - | | | | |
| natural henna | | | | |
| based powder | | | | |
| Example 1 | 12% | 5.2% | 82.8% | 1% |
| (SCPF 1) | | | | |
| Example 2 | 12% | 4.1% | 83.9% | 1% |
| (SCPF 2) | | | | |
| Example 3 | 12% | 4.1% | 83.9% | 1% |
| (SCPF 3) | | | | |
| Example 4 | 12% | 8.1% | 79.8% | 1% |
| (SCPF 4) | | | | |

Results of tests carried out on Caucasian slightly bleached hair are listed in Table 3. Lower wet combing times indicate good conditioning of the fibre, such as for Examples 1, 2 and 3. Examples 1, 2 and 4 have lower colour loss compared to Comparative example 1 and Example 3. Examples 3 and 4 are shinier than Comparative Example 1.

TABLE 3

| Tests on Caucasian | | | | |
|---|---|---|---|---|
| slightly bleached hair | Wet combing | Sensory | Shine | Colourloss |
| Comparative | 36 seconds | Reference | Reference | ΔE = 4.68 |
| Example 1 - natural | | | | |
| henna based powder | | | | |
| Example 1 (SCPF 1) | 19 seconds | Easier to comb | No significant | ΔE = 3.77 |
| | | than the reference | difference | |
| | | | observed | |
| Example 2 (SCPF 2) | 24 seconds | No significant | No significant | ΔE = 3.66 |
| | | difference | difference | |
| | | observed | observed | |
| Example 3 (SCPF 3) | 14 seconds | Easier to comb | Shinier than | ΔE = 4.41 |
| | | than the reference | the reference | |
| Example 4 (SCPF 4) | 29 seconds | No significant | Shinier than | ΔE = 3.62 |
| | | difference | the reference | |
| | | observed | | |

### Examples 5 TO 7 and Comparative Example 2

The natural henna based powder described above was mixed with SCPF produced in Preparation Example 1 in the amounts as disclosed in Table 4, using the following procedure:
Blend the natural henna powder hair colourant and silicone compositions in powder form together at amount disclosed in Table 4
Add water @37° C. and mix with the spatula
Soak this preparation in water for 15 minutes
Apply 2 g of mixture per hair tress
Leave on hair during 30 minutes
Rinse each tress with water @37° C. during 1 min 30 s
For detangling test: proceed to the evaluation and let dry for 1 night for other testings
For other uses: let dry for 1 night

TABLE 4

| | | | | Silicone |
|---|---|---|---|---|
| | Colourant | SCPF | Water | active |
| | content | content | content | content |
| Comparative | 12.5% | 0% | 87.5% | 0% |
| Example 2 - natural | | | | |
| henna based | | | | |
| powder | | | | |
| Example 5 (SCPF 5) | 12.5% | 5% | 82.5% | 1% |
| Example 6 (SCPF 6) | 12.5% | 5% | 82.5% | 1% |
| Example 7 (SCPF 7) | 12.5% | 5% | 82.5% | 1% |

Results of tests carried out on Indian hair are listed in Table 5. Examples 5, 6 and 7 are easier to comb than Comparative Example 2. Examples 5 and 6 have improved feel over Comparative Example 2 and Example is shinier.

TABLE 5

| Tests on | Wet | | | |
|---|---|---|---|---|
| Indian hair | combing | Sensory | Shine | Colourloss |
| Comparative | 6.3 | Reference | Reference | ΔE = 0.21 |
| Example 2 - | seconds | | | |
| natural | | | | |
| henna based | | | | |
| powder | | | | |
| Example 5 | 1.6 | Smoother than | Duller than the | ΔE = 2.4 |
| (SCPF 5) | seconds | reference | reference | |
| Example 6 | 1.4 | Easier to comb | Duller than the | ΔE = 0.19 |
| (SCPF 6) | seconds | than reference | reference | |
| Example 7 | 4.2 | No significant | Shinier than the | ΔE = 0.16 |
| (SCPF 7) | seconds | difference | reference | |
| | | observed | | |

### Example 8 and Comparative Example 3

The bleaching agent powder described above was mixed with SCPF12 produced in Preparation Example 1 in the amounts as disclosed in Table 6 and applied to cotton towels for a bleaching trial, using the following procedure:
1° Blend bleaching powder and silicone compositions in powder form together
2° Add water at 40° C. and mix with a spatula
3° Soak cotton towels during 2 hours
4° Rinse each towel with tap water at 20° C.
5° Leave the towels to dry for one day

TABLE 6

| | | | | Silicone |
|---|---|---|---|---|
| | Bleaching | SCPF | Water | active |
| | content | content | content | content |
| Comparative example 3: | 0.5% | 0% | 99.5% | 0% |
| commercial bleaching powder | | | | |
| “Eau écarlate” | | | | |
| Example 8: commercial | 0.5% | 0.5% | 99% | 0.05% |
| bleaching powder | | | | |
| “Eau écarlate” + SCPF 12 | | | | |

The colour of the cotton towels was measured before and after the application of the bleaching compositions. The softness of the towels was evaluated by 16 panellists.

Towels treated with Comparative example 3 showed a colour loss (Delta E) of 14.42, and towels treated with Example 8 showed a colour loss of 14.95. This indicates that the addition of the silicone composition does not impact the quality of bleaching occurring.

On the contrary to the softness parameter, for which 11 out of the 16 panellists rated the towels treated with Example 8 softer than those treated with Comparative example 3.

1. A solid coloring composition comprising
(A) a solid color modifying composition
(B) a silicone in powder form
wherein the solid color modifying composition (A) comprises at least one color modifying agent such as a synthetic dye, a natural dye, a bleaching agent, or a mixture thereof.

2. (canceled)

3. The composition of claim 1 where the color modifying agent is selected from synthetic dyes, natural dyes or mixtures thereof.

4. The composition of claim 1 where the color modifying agent is a bleaching agent.

5. The composition of as claimed in claim 1 where (B) comprises a silicone polymer, a carrier and optionally a binder.

6. The composition as claimed in claim 1 where the silicone polymer in (B) is selected from silicone oils, silicone waxes, silicone resins, silicone gums and silicone polymers modified with organofunctional groups.

7. The composition of as claimed in claim 1 wherein the silicone polymer in (B) is selected from polyalkylsiloxanes containing trimethylsilyl end groups, polyalkylsiloxanes containing dimethylsilanol end groups, silicone polymers modified with organofunctional groups such as phenyl, amine groups, polyether groups, quaternary ammonium groups, saccharide groups, amino acids, vinyl groups, hydroxyl groups; and mixtures thereof

8. The composition as claimed in claim 1 where the silicone polymer in (B) is an emulsion of dimethicone, a dimethiconol, an amino functional silicone or a divinyldimethicone/dimethicone copolymer.

9. The composition as claimed in claim 1 where the weight ratio of silicone polymer to carrier in (B) is in the range 2:98 to 40:60

10. The composition as claimed in claim 1 where (B) has a mean particle size in the range of 0.02 to 1.50 mm.

11. A method of making a solid coloring composition comprising the steps of
a. Producing a silicone in powder form by contacting the liquid silicone containing composition with the carrier in a mixer in which droplets of the liquid silicone containing composition become agglomerated with the carrier
b. Combining the silicone in powder form (B) with a solid color modifying composition (A) wherein the solid color modifying composition (A) comprises at least one color modifying agent such as a synthetic dye, a natural dye, a bleaching agent, or a mixture thereof.

12. The method of claim 11 where the ratio of (B) to (A) in the solid coloring composition is in the range of 1:99 to 90:10.

13. A method of modifying the color of fiber comprising the steps of
a. Providing a solid coloring composition as claimed in claim 1
b. Mixing the solid coloring composition with water to form a water mixture
c. Applying the water mixture to a fiber
d. Rinsing the fiber.

14. The method of claim 13 where the ratio solid coloring composition to water is in the range of 1:5 to 1:12.
